(19) United States
(12) Patent Application Publication
(10) Pub. No.: US 2003/0103888 A1
(43) Pub. Date: Jun. 5, 2003

(54) Granular zirconium phosphate and methods for synthesis of same

(76) Inventors: Ton That Hai, Round Lake, IL (US); Paul Sanders, Greendale, WI (US); Mark Nordhaus, Mundelein, IL (US); Sujatha Karoor, Lake Bluff, IL (US); Cong Jiang, Gurnee, IL (US); Ben Melnick, Chicago, IL (US)

Correspondence Address: BAXTER HEALTHCARE CORPORATION, RENAL DIVISION, 1 BAXTER PARKWAY, DF3-3E, DEERFIELD, IL, 60015, US

(21) Appl. No.: 09/993,737

(22) Filed: Nov. 13, 2001

(51) Int. Cl. C01B 25/37; C01B 15/16

(52) U.S. Cl. 423/308; 423/311

(57) ABSTRACT

Compositions including zirconium phosphate particles as well as methods of synthesizing and using same are provided. The zirconium phosphate particles are synthesized through the use of polyphosphate and zirconyl chloride.

### BACKGROUND OF THE INVENTION

The present invention relates generally to zirconium containing materials. More specifically, the present invention relates to zirconium phosphate based materials that can be used to absorb ammonia.

Zirconium phosphate is typically produced by two or three different methods. One such method is a reflux method as proposed by Clearfield and Stynes in A. Clearfield and J. A. Stynes, The Preparation of Crystalline Zirconium Phosphate and Some Observations on its Ion Exchange Behavior, J. Inorg. Nucl. Chem., 26, 117-119 (1964). A second method is by direct precipitation as proposed by Alberti and Torracca in G. Alberti and E. Torracca, Crystalline Insoluble Salts of Polybasic Metals, Synthesis of Crystalline Zirconium and Titanium Phosphate by Direct Precipitation, J. Inorg. Nucl. Chem., 30, 317-319 (1968). Lastly, Marantz et al. have reported in U.S. Pat. No. 3,850,835 the production of zirconium phosphate by adding zirconyl powder to an orthophosphoric acid solution.

Pursuant to the Clearfield method zirconium phosphate is formed as a gel. This is followed by refluxing the gel form in highly concentrated phosphoric acid for several days. This converts the gel into macroscopic crystalline zirconium phosphate. The Clearfield method requires a multiple step synthesis. Due to the time involved in the heating process the Clearfield method is an expensive process and therefore the resultant product is expensive.

The Alberti method involves a direct precipitation using hydrofluoric acid. In this regard, hydrofluoric acid is used to dissolve the zirconium salts. To this end, zirconium salt is placed in a solution of hydrofluoric acid to which is added phosphoric acid. A soluble zirconium phosphate complex results. The hydrofluoric acid is then removed through evaporation to produce crystalline zirconium phosphate. A disadvantage of this method is that hyrdrofloric acid is very toxic. Furthermore, the resultant crystalline zirconium is very fine and therefore cannot be used in many methods or applications for zirconium such as ion exchange chromatography columns, e.g., the particles will pack too tightly together in the column.

The Marantz method also has a number of disadvantages. The zirconium phosphate product produced in Marantz does not contain uniform particles. Furthermore, a number of small particles are present. This can create back pressure problems that prevent the use of the product in a number of applications, for example, in an ion exchange column.

Granular zirconium phosphate can be used for a number of purposes. One such use is in a chromatography column for ion exchange. Zirconium phosphate can also be used as a part, or as an entire, resin bed to absorb ammonium ions, calcium and magnesium. In this regard, ammonium ions can be removed from a solution via an ion exchange process using zirconium phosphate. Zirconium phosphate contains two counter ions, hydrogen and sodium. The release of the counter ions is determined, in part, by the solution pH and the resin.

It has also been determined, that the absorption characteristics of zirconium phosphate vis-à-vis ammonia absorption is due, in part, to the particle size and distribution of the zirconium phosphate. Heretofore, prior art methods produced an irregular particle distribution. In fact, for some applications, currently available zirconium phosphate may not provide an entirely satisfactory product.

Therefore, there is a need for an improved method of producing zirconium phosphate and zirconium phosphate so produced.

### SUMMARY OF THE INVENTION

The present invention provides improved zirconium phosphate compositions as well as methods of making and using same. Zirconium phosphate compositions of the present invention have a better particle distribution than the prior art. Moreover, the composition will provide for a better absorption of materials such as ammonia, calcium, and magnesium.

To this end, in an embodiment, the present invention provides a composition comprising granules of zirconium phosphate synthesized with polyphosphate and zirconyl chloride under certain processing conditions.

In an embodiment, the polyphosphate is selected from the group consisting of sodium hexametaphosphate, sodium trimetaphosphate, sodium tripolyphosphate, and sodium pyrophosphate.

In an embodiment, the granules have a crystal structure.

In another embodiment of the present invention, a composition comprising zirconium phosphate particles is provided. The particles having a size distribution such that 97% of the particles have a size of greater than 4 μm and 99% of the particles have a size of less than 100 μm; 97% of the particles have a size of greater than 1 μm and 99% of the particles has a size of less than 250 μm.

In an embodiment, the zirconium phosphate is obtained through a synthesis process using a polyphosphate.

In a further embodiment of the present invention, a method of preparing zirconium phosphate particles is provided. The method comprising the steps of: adding zirconyl chloride to a polyphosphate solution; and heating a resultant solution or mixture to obtain zirconium phosphate particles.

In an embodiment of the method the solution or mixture is reduced by heating under reflux the resultant solution or mixture produced by the addition of zirconyl chloride to polyphosphate.

In an embodiment of the method the zirconium phosphate particles are purified by a washing step.

In an embodiment of the method the pH of the solution is between 3.0 and 6.0, the polyphosphate concentration is between 0.1M and 1.5M, and the polyphosphate/zirconyl chloride molar ratio is between 1/1 to 10/1.

In yet another embodiment of the present invention, a composition of zirconium phosphate particles prepared from sodium pyrophosphate is provided. The particles having a particle distribution such that at least 97% of the particles have a size of greater than 15 μm; at least 90% of the particles have a size of greater than 20 μm; at least 75% of the particles have a size of greater than 25 μm; at least 50% of the particles have a size of greater than 30 μm; at least 25% of the particles have a size of greater than 35 μm; at least 1% of the particles have a size of greater than 70 μm.

In still a further embodiment of the present invention, a composition of zirconium phosphate particles prepared from sodium triphosphate is provided. The particles having a particle distribution such that at least 97% of the particles have a size of greater than 4 μm; at least 90% of the particles have a size of greater than 10 μm; at least 75% of the particles have a size of greater than 20 μm; at least 50% of the particles have a size of greater than 25 μm; at least 25% of the particles have a size of greater than 35 μm; at least 1% of the particles have a size of greater than 80 μm; and at least 97% of the particles have a size of greater than 1 μm; at least 90% of the particles have a size of greater than 4 μm; at least 75% of the particles have a size of greater than 10 μm; at least 50% of the particles have a size of greater than 20 μm; at least 25% of the particles have a size of greater than 30 μm; at least 1% of the particles have a size of greater than 105 μm.

In another embodiment of the present invention, a composition for removing ammonia from a fluid stream comprising particles of zirconium phosphate synthesized using polyphosphate and zirconyl chloride is provided.

Yet further, in an embodiment of the present invention, a particle bed for removing ammonium from a fluid stream is provided. The particle bed comprising zirconium phosphate particles having a size distribution such that 97% of the particles have a size of greater than 4 μm and 99% of the particles have a size of less than 100 μm; 97% of the particles have a size of greater than 1 μm and 99% of the particles has a size of less than 250 μm.

In a still further embodiment of the present invention a method of providing dialysis is provided. The method comprising the step of passing a dialysate fluid through a particle bed including a composition comprising granules of zirconium phosphate synthesized with polyphosphate and zirconyl chloride using certain processing conditions.

An advantage of the present invention is to provide an improved method of synthesizing zirconium phosphate.

Still further, an advantage of the present invention is to provide improved zirconium phosphate compositions.

Moreover, an advantage of the present invention is to provide a zirconium phosphate material that can be used in a column.

Additionally, an advantage of the present invention is to provide an improved composition for absorbing products, such as ammonia, from a fluid stream.

Another advantage of the present invention is to provide an improved resin for use in a device for removing a selected product from a fluid stream.

Still, an advantage of the present invention is to provide an improved product that can be used in medical procedures.

Furthermore, an advantage of the present invention is to provide an improved zirconium phosphate composition that can be used in a dialysis method.

Additional features and advantages of the present invention will be described in and apparent from the detailed description of the detailed description of the invention and the figures.

### BRIEF DESCRIPTION OF THE FIGURES

FIGS. 1a and 1b illustrate graphically typical size distribution of zirconium phosphate particles prepared from the Reaction Type 1; FIG. 1a is without sonication and FIG. 1b is with one minute of sonication.

FIGS. 2a and 2b illustrate typical photomicrograph of zirconium phosphate particles prepared from the Reaction Type 1; FIG. 2a is taken at 90× magnification and FIG. 2b at 500×.

FIGS. 3a and 3b illustrate graphically typical size distribution of zirconium phosphate particles prepared from the Reaction Type 2; FIG. 3a is without sonication and FIG. 3b is with one minute of sonication.

FIGS. 4a and 4b illustrate typical photomicrograph of zirconium phosphate particles prepared from the Reaction Type 2; FIG. 4a is taken at 90× magnification and FIG. 4b at 500×.

### DETAILED DESCRIPTION OF THE INVENTION

The present invention provides improved zirconium phosphate compositions as well as methods of synthesizing same. Pursuant to the present invention zirconium phosphate is produced that has a better particle distribution than products that have been available heretofore. Moreover, the present invention provides methods of synthesizing zirconium phosphate that are more commercially viable and/or safer.

Pursuant to the present invention, in an embodiment, zirconium phosphate is obtained that has a high absorption capacity for ammonia, magnesium, and calcium. The methods of the present invention provide for a good yield through an inexpensive process. The zirconium phosphate is obtained as large granular particles. These particles may or may not have a uniform crystal structure. However, in a preferred embodiment, large uniform crystals are produced.

Although in the embodiment of the present invention set forth below, the zirconium phosphate is used to remove ammonia from dialysis solutions in artificial kidney systems, i.e., ammonia produced from the decomposition of urea by urease, the zirconium phosphate can be used in a variety of other products. For example, the zirconium phosphate can be used as a stationary phase for ion exchange chromatography.

Pursuant to the present invention, zirconium phosphate is produced using a polyphosphate. The polyphosphates that can be used include: sodium hexametaphosphate; sodium trimetaphosphate; sodium tripolyphosphate; and sodium pyrophosphate. The polyphosphate is combined with zirconyl chloride to produce zirconium phosphate under controlled reaction conditions of pH, polyphosphate concentration and polyphosphate/zirconyl chloride molar ratio. It should be noted that other zirconium salts can be used including zirconium sulfate and zirconyl nitrate.

In this regard, in a first step of the process a gel is formed by the addition of a polyphosphate to zirconyl chloride. The reaction is carried out at a suitable pH, polyphosphate concentration and polyphosphate/zirconyl chloride molar ratio. The pH of the reaction can range from approximately 3.0 to 6.0. Preferable pHs range from approximately 3.8 to about 5.7 and 3.6 to about 5.5 before addition of zirconyl chloride and before refluxing, respectively. The polyphosphate concentration can range from approximately 0.1 M to 1.5 M. Preferable polyphosphate concentrations range from approximately 0.5 M to about 1.25 M. The polyphosphate/zirconyl chloride molar ratio can range from approximately 1/1 to about 10/1. Preferable polyphosphate/zirconyl chloride molar ratios range from approximately 3/1 to about 5/1. At these reaction conditions the zirconium phosphate gel gradually dissolves in the reaction medium.

In a second step in the synthesis process, the mixture from the first step is heated under reflux. Preferably the clear reaction mixture is heated at a temperature of approximately 90° C. to about 105° C. Preferably the mixture is heated for approximately 6 to about 24 hours. This heating step reduces the mixture, and specifically the precipitation to large particle sizes of zirconium phosphate. If the resultant gel from the reaction of polyphosphate and zirconyl chloride is not completely soluble in the reaction medium, the product obtained after refluxing has a broader distribution of particles.

If desired, the particles can be isolated and purified. A method of isolating and purifying the particles is by washing with water using a vacuum filtration and/or decantation method. Preferably, the washing step is performed at a pH of at least 8.0 by adjusting the pH of a slurry containing the particles.

The method of the present invention provides a variety of advantages over the prior art. Thus, the product of the present invention can be produced in an inexpensive manner. Further, the process of the present invention does not require the use of toxic compounds such as hyrdofluoric acid.

One of the unexpected advantages of the present invention is that far more uniform particles are obtained than with prior art methods. These uniform particles provide improved absorption characteristics and elimination of back pressure issues. In this regard, in an embodiment the product of the present invention has a particle distribution such that: approximately 90% of particles have a size of greater than 10 μm; approximately 75% of particles have a size of greater than 20 μm; approximately 50% of particles have a size of greater than 25 μm; approximately 25% of particles have a size of greater than 35 μm; approximately 1% of particles have a size of greater than 70 μm.

Set forth below in the examples are experiments demonstrating advantages of the zirconium phosphate product over the prior art. These advantages include an increase in ammonia absorption capacity that is greater than the prior art. It has been found that the particles of the present invention have an ammonia capacity of at least 0.8 and typically 0.8-1.4 mmol/g using an ammonia fed of approximately 7000 μmol/l.

In an embodiment, the zirconium phosphate of the present invention is utilized in a dialysis process e.g., a continuous flow peritoneal dialysis procedure. In this regard, zirconium phosphate can be used in a cartridge for removing uremic toxins in a dialysis process. Such a cartridge as well as methods of using same are disclosed in U.S. patent application Ser. No. ______ entitled “Method and Composition for Removing Uremic Toxins in Dialysis Processes” which is being filed herewith, the disclosure of which is herein incorporated by reference.

By way of example and not limitation, examples of the present invention will now be given.

### EXAMPLE NO. 1

A determination of ammonia sorption capacity was performed using a dynamic test system.

The following materials were used:

1. Column: BioRad Bio-Scale Column.

2. Pump: Applied Biosystems, Model 400 Solvent Delivery System.

3. Fraction Collector: Isco, Model Retriever III.

4. Solution Matrix: Baxter, Dianeal PD-1 Dialysis Solution.

5. Ammonium Chloride: Mallinckrodt, Granular, AR, ACS, 99.5%.

The BioRad Bio-Scale column was packed with the test article (i.e., zirconium phosphate) per BioRad directional insert. The products that were tested are described below. A mobile phase consisting of Baxter Dianeal PD-1 solution spiked with 7000 umol/L ammonium chloride, was then pumped through the column at 1 mL/min. over 200 minutes. Fractions (2 mL) were collected at time points 0, 2, 4, 6, 8, 10, 20, 30, 40, 50, 60, 70, 80, 90, 100, 110, 120, 130, 140, 150, 160, 170, 180, 190, and 200 minutes. The collected fractions were then analyzed for Ca, Phosphorous, Mg, Na, NH₃, and pH using clinical chemistry analyzers.

The ammonia capacity was calculated as follows:

$$\frac{\begin{matrix}[ NH3 ] {\text{(mmol/L)}}_{Feed} \times Flow Rate \text{(ml/min.)} \times \\ B . T . \text{(min.)} \times 0.001 \times \text{L/ml}\end{matrix}}{ZP \text{(g)}} = Ammonia Capcity \text{(mmol/g)}$$

Where: [NH3] (mmol/L)_{Feed} is the ammonia concentration in the mobile phase, Flow Rate (ml/min.)=rate at which the mobile phase is pumped, B.T. (min.)=The time point (fraction) at which the ammonia concentration exceeds 1.0 mmol/L, and ZP=weight of zirconium phosphate packed in the column.

The results of the analysis are set forth in Tables 1-4 for ammonia sorption capacity. Calcium (3.5 mEq/L) and magnesium (1.5 mEq/L) are completely absorbed by the zirconium phosphate column.

### EXAMPLE NO. 2

Zirconyl chloride octahydrate (32.2 g, 100 mmol) was added as a powder to a solution of sodium tripolyphosphate (pentasodium salt, 36.78 g, 100 mmol) in water (180 mL) containing 5N HCl (20 mL, 100 mmol). The mixture was stirred at room temperature for 5 hours, during this time complete dissolution of precipitate was not observed. The reaction mixture was heated to reflux overnight. After cooling to room temperature, the precipitate was isolated by filtration, and washed in a sequence of repeated cycles (8 cycles) involving redispersion in deionized water (850 mL) and vacuum filtration to remove chloride ion (tested with 1 M silver nitrate). The washed precipitate was stirred in water (200 mL) and the pH of the mixture was adjusted to 7.46 from its original value of 6.52 using 9% sodium bicarbonate solution. The precipitate was collected by vacuum filtration and washed with water (2×300 mL) and dried under vacuum at room temperature. Product Z-STP-7 (26.6 g) was obtained.

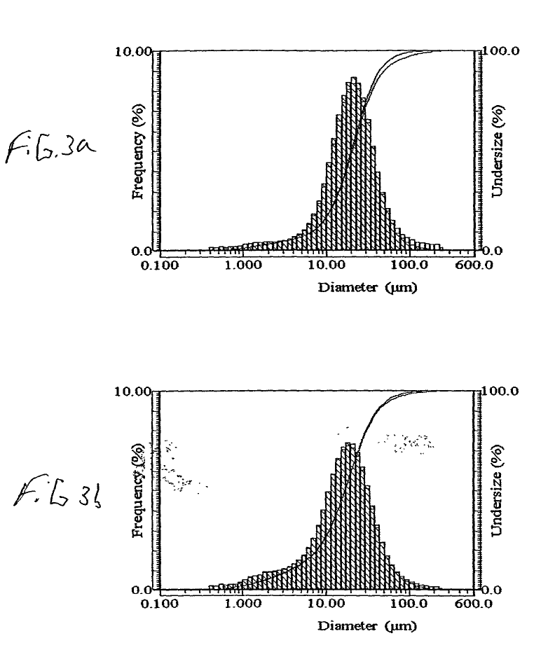

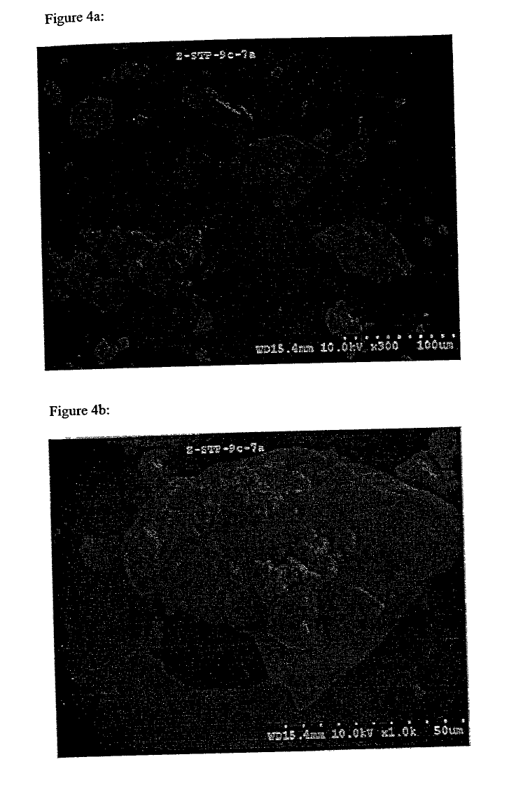

Other reaction conditions and characteristics of zirconium phosphate obtained from sodium tripolyphosphate are summarized in Tables 1, 2, and 5 and FIGS. 3-4.

TABLE 1
Synthesis of Zirconium Phosphate using Sodium Tripolyphosphate

| | STP^{b} | Water | 5N HCl mL | ZrOCl₂.8H₂O | pH^{c} | | Yield |
|---|---|---|---|---|---|---|---|
| Product^{a} | g (mol) | (mL) | (mol) | g (mol) | pH I | pH II | (g) |
| Z-STP-1 | 184 (0.5) | 1020 | 100 (0.5) | 32.2 (0.1) | 5.82 | 5.35 | 26.6 |
| Z-STP-2 | 45.9 | 222.5 | 27.5 (0.1375) | 8.06 (0.025) | 5.43 | 5.05 | 2.2 |
| | (0.125) | | | | | | |
| Z-STP-3 | 45.9 | 215 | 35 (0.175) | 8.06 (0.025) | 4.77 | 4.24 | 4.8 |
| | (0.125) | | | | | | |
| Z-STP-4^{d} | 147.2 (0.4) | 720 | 80 (0.4) | 32.2 (0.1) | 5.98 | 5.49 | 26.1 |
| Z-STP-5^{d} | 55.2 (0.15) | 270 | 30 (0.15) | 16.1 (0.05) | 5.60 | 5.02 | 14.3 |
| Z-STP-6^{e} | 73.6 (0.2) | 360 | 40 (0.2) | 32.2 (0.1) | 5.85 | 4.80 | 28.2 |
| Z-STP-7^{e} | 36.8 (0.1) | 180 | 20 (0.1) | 32.2 (0.1) | 5.57 | 2.77 | 26.6 |

^{a}Z-STP is designated as the name of zirconium phosphate prepared from sodium tripolyphosphate.
^{b}STP: Sodium tripolyphosphate, pentasodium salt.
^{c}pH: pH I: pH of reaction mixture before addition of ZrOCl₂.8H₂O.
pH: pH II: pH of reaction mixture after addition of ZrOCl₂.8H₂O and before refluxing.
^{d}The reaction mixture was turbid before refluxing but became clear after refluxing for 0.5-1 hours.
^{e}Gel formed from the reaction of ZrOCl₂.8H₂O with STP was partially soluble in the reaction medium before refluxing.

TABLE 2
Characteristics of Zirconium Phosphate obtained from Sodium
Tripolyphosphate

| | Ammonium | Ammonium | | |
|---|---|---|---|---|
| Zirconium | Feed | Capacity | | P/Zr^{c} |
| Phosphate^{a} | (umol/L) | (mmol/g) | pH^{b} | Molar Ratio |
| Z-STP-2 | 6780 | 0.90 | 8.4, 7.3 | 1.92 |
| Z-STP-3 | 7210 | 0.93 | 8.4, 7.9 | 1.79 |
| Z-STP-4 | 7500 | 0.80 | 7.6, 6.8 | 1.86 |
| Z-STP-5 | 7080 | 0.56 | 7.4, 6.4 | 1.98 |
| Z-STP-6 | 7330 | 1.02 | 8.0, 7.0 | 1.83 |
| Z-STP-7 | 6860 | 0.72 | 6.4, 6.2 | 1.94 |

^{a}Z-STP is designated as the name of zirconium phosphate obtained from sodium tripolyphosphate.
^{b}pH of eluent (Dianeal Solution) at time zero, and at time of ammonia breakthrough respectively.
^{c}The P/Zr molar ratio was determined by ICP-AES method (Inductive coupling plasma-atomic emission spectroscopy method)

### EXAMPLE NO. 3

Zirconyl chloride octahydrate (32.2 g, 100 mmol) was added as a powder to a solution of sodium tripolyphosphate (pentasodium salt, 184 g, 500 mmol) in water (1.02 L) containing 5N HCl (100 mL, 500 mmol). After stirring at room temperature for 5 hours, the reaction mixture was completely clear. Reaction mixture was heated to reflux overnight to induce the precipitation of product. The precipitate was isolated by filtration, and washed in a sequence of repeated cycles (9 cycles) involving redispersion in deionized water (800 mL) and vacuum filtration to remove chloride ion. The washed precipitate was stirred in water (200 mL) and the pH of the mixture was adjusted to 7.45 from its original value of 7.0 (pH was determined by pH paper) using 9% sodium bicarbonate solution. The precipitate was collected by vacuum filtration and washed with water (2×300 mL) and dried under vacuum at room temperature. Product Z-STP-1 (26.6 g) was obtained.

### EXAMPLE NO. 4

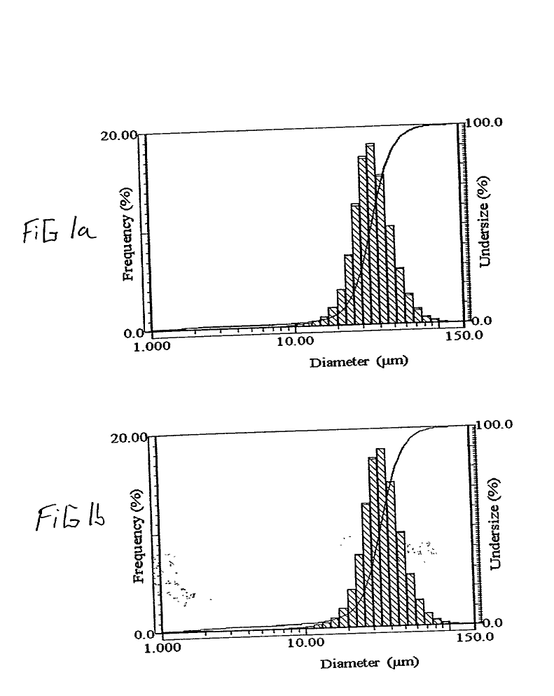

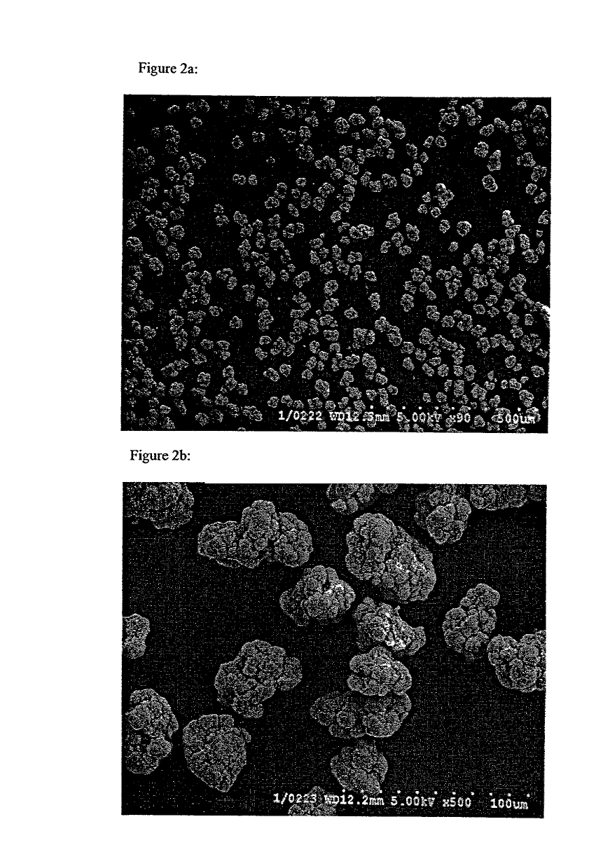

Typical zirconium phosphate obtained from zirconium sodium pyrophosphate was prepared as follows. Zirconyl chloride octahydrate (64.5 g, 200 mmol) was added as a powder to a solution of sodium pyrophosphate (tetrasodium salt, 446 g, 500 mmol) in water (700 mL) containing 5N HCl (300 mL, 1.5 mol). The mixture was stirred at room temperature until a slightly turbid solution was obtained (3 hours). The reaction mixture was heated to reflux overnight. Heavy white precipitates were formed during refluxing. The mixture was cooled to room temperature and supernatant was decanted. Water (600 mL) was added to the solid, and stirred vigorously for 5-10 minutes. The solid was collected by vacuum filtration, and the resulting filter cake washed with water (2×300 mL). The filter cake was dispersed in 600 mL of water and stirred vigorously, allowed to settle and decanted off the supernatant. This process was repeated 8 additional times. The mixture was filtered and the filter cake was washed 2×300 mL with water. The filter cake was dispersed in 400 mL of water, and the slurry pH was adjusted with 1N NaOH to a target of 8.5-9.0 (pH was determined by pH paper). The slurry was filtered and the filter cake was washed with 300 mL of water. The filter cake was dispersed in 600 mL of water and stirred for 5-10 minutes. The slurry was filtered again and washed with 300 mL of water. The filter cake was left on the filter under hose vacuum overnight. The white solid was dried under high vacuum for 15-24 hours to get 60 g of zirconium phosphate. Other reaction conditions and characteristics of the zirconium phosphate obtained from sodium pyrophosphate are summarized in Tables 3, 4, and 5 and FIGS. 1 and 2.

TABLE 3
Synthesis of Zirconium Phosphate using Sodium Pyrophosphate

| | SPP^{b} | Water | 5N HCl mL | ZrOCl₂.8H₂O | pH^{c} | | Yield |
|---|---|---|---|---|---|---|---|
| Product^{a} | g (mol) | (mL) | (mol) | g (mol) | pH I | pH II | (g) |
| Z-SPP-1 | 446 (1) | 700 | 300 (1.5) | 64.5 (0.2) | 5.20 | 3.73 | 60 |
| Z-SPP-2 | 446 (1) | 530 | 300 (1.5) | 64.5 (0.2) | 5.30 | 3.80 | 62 |
| Z-SPP-3 | 357 (0.8) | 560 | 240 (1.2) | 64.5 (0.2) | 5.28 | 3.75 | 50 |
| Z-SPP-4 | 357 (0.8) | 424 | 240 (1.2) | 64.5 (0.2) | 5.20 | 3.70 | 59 |
| Z-SPP-5 | 268 (0.6) | 600 | 150 (0.75) | 64.5 (0.2) | 5.67 | 3.86 | 57 |

^{a}Z-SPP is designated as the name of zirconium phosphate prepared from sodium pyrophosphate.
^{b}SPP: Tetrasodium pyrophosphate, decahydrate.
^{c}pH: pH I: pH of reaction mixture before addition of ZrOCl₂.8H₂O.
pH: pH II: pH of reaction mixture after addition of ZrOCl₂.8H₂O and before refluxing.

TABLE 4
Characteristics of Zirconium Phosphate Obtained from Sodium
Pyrophosphate

| | Ammonium | Ammonium | | |
|---|---|---|---|---|
| Zirconium | Feed | Capacity | | P/Zr^{c} |
| Phosphate^{a} | (umol/L) | (mmol/g) | pH^{b} | Molar Ratio |
| Z-SPP-1 | 6890 | 1.23 | 8.7, 7.3 | 1.83 |
| Z-SPP-2 | 6800 | 1.28 | 8.6, 7.7 | 1.95 |
| Z-SPP-3 | 6770 | 1.34 | 8.5, 7.8 | 1.93 |
| Z-SPP-4 | 7220 | 1.24 | 8.0, 7.5 | 1.87 |
| Z-SPP-5 | 7000 | 1.39 | 8.3, 7.9 | 1.89 |

^{a}Z-SPP designates the zirconium phosphate prepared from sodium pyrophosphate.
^{b}pH of eluent (Dianeal Solution) at time zero, and at time of ammonia breakthrough respectively.
^{c}The P/Zr molar ratio was determined by ICP-AES method (Inductive coupling plasma-atomic emission spectroscopy method).

TABLE 5
Particle Size Distribution of Zirconium Phosphate Prepared
from STP or SPP^{a}

| | | | Mean | Standard | | | | | | |
|---|---|---|---|---|---|---|---|---|---|---|
| Zirconium | Reaction | Sonica- | Diameter | Deviation | 97%^{d} | 90%^{e} | 75%^{f} | 50%^{g} | 25%^{h} | 1%ⁱ |
| Phosphate | Type^{b} | tion^{c} | (μm) | (μm) | (μm) | (μm) | (μm) | (μm) | (μm) | (μm) |
| Z-SPP-1 | 1 | No | 34.839 | 10.283 | 20.18 | 23.89 | 27.98 | 33.20 | 39.56 | 70.28 |
| | | Yes | 34.832 | 10.368 | 20.13 | 23.83 | 27.93 | 33.16 | 39.56 | 70.84 |
| Z-SPP-3 | 1 | No | 36.729 | 13.242 | 15.22 | 23.06 | 28.68 | 35.33 | 43.26 | 79.24 |
| | | Yes | 36.110 | 13.537 | 11.33 | 22.14 | 28.15 | 34.91 | 42.87 | 78.68 |
| Z-SPP-5 | 1 | No | 42.444 | 13.556 | 23.37 | 28.17 | 33.5 | 40.18 | 48.73 | 99.68 |
| | | Yes | 41.982 | 13.270 | 23.1 | 27.89 | 33.19 | 39.85 | 48.28 | 87.51 |
| Z-STP-1 | 1 | No | 29.185 | 15.468 | 4.62 | 13.27 | 20.00 | 27.19 | 35.80 | 84.59 |
| | | Yes | 27.452 | 14.919 | 2.15 | 10.84 | 18.4 | 25.94 | 34.33 | 77.90 |
| Z-STP-4 | 2 | No | 28.582 | 34.851 | 1.83 | 6.93 | 12.75 | 20.44 | 31.76 | 207.71 |
| | | Yes | 22.438 | 23.854 | 1.28 | 3.63 | 9.36 | 16.87 | 27.55 | 129.11 |
| Z-STP-5 | 2 | No | 46.212 | 68.956 | 1.84 | 4.98 | 12.33 | 21.73 | 42.19 | 343.09 |
| | | Yes | 28.751 | 52.267 | 1.52 | 3.00 | 8.14 | 14.98 | 25.22 | 297.39 |
| Z-STP-7 | 2 | No | 26.250 | 20.618 | 1.77 | 7.56 | 12.79 | 20.97 | 33.76 | 105.56 |
| | | Yes | 23.613 | 19.309 | 1.39 | 5.53 | 10.57 | 18.34 | 31.42 | 95.20 |

^{a}Size distribution of particles was determined by scanning electron microscopy and through the use of laser diffraction technology.
^{b}Reaction type 1: The gels formed from the reaction of zirconyl chloride with STP or SPP are completely soluble in the reaction medium before refluxing. Reaction type 2: The gels formed from the reaction of zirconyl chloride and STP or SPP are not completely soluble in the reaction medium before refluxing.
^{c}Sonicated for 1 minute.
^{d}97% of particles have the particle size (μm) greater than the value indicated in the column, e.g. 97% of Z-SPP-1 have particle size greater than 20.18 μm.
^{e}90% of particles have the particle size (μm) greater than the value indicated in the column, e.g. 90% of Z-SPP-1 have particle size greater than 23.89 μm.
^{f}75% of particles have the particle size (μm) greater than the value indicated in the column, e.g. 75% of Z-SPP-1 have particle size greater than 27.98 μm.
^{g}50% of particles have the particle size (μm) greater than the value indicated in the column, e.g. 50% of Z-SPP-1 have particle size greater than 33.20 μm.
^{h}25% of particles have the particle size (μm) greater than the value indicated in the column, e.g. 25% of Z-SPP-1 have particle size greater than 39.56 μm.
ⁱ1% of particles have the particle size (μm) greater than the value indicated in the column, e.g. 99% of Z-SPP-1 have particle size greater than 70.28 μm.

It should be understood that various changes and modifications to the presently preferred embodiments described herein will be apparent to those skilled in the art. Such changes and modifications can be made without departing from the spirit and scope of the present invention and without diminishing its intended advantages. It is therefore intended that such changes and modifications be covered by the appended claims.

The invention is claimed as follows:

1. A composition comprising zirconium phosphate granules synthesized using polyphosphate and zirconyl chloride under conditions wherein the pH of a mixture of polyphosphate and zinconyl chloride is at least 3.0 and the mixture is heated to greater than ambient conditions.

2. The composition of claim 1 wherein the polyphosphate is selected from the group consisting of sodium hexametaphosphate, sodium trimetaphosphate, sodium tripolyphosphate, and sodium pyrophosphate.

3. The composition of claim 1 wherein the granules have a crystal structure.

4. The composition of claim 1 wherein the granules have a phosphate to a zirconium molar ratio of approximately 1.8/1 to about 2.2/1.

5. The composition of claim 1 wherein the granules have an ammonia sorption capacity of at least 0.8 mmol/g using an ammonia feed of approximately 7000 μmo/l.

6. A composition comprising zirconium phosphate particles having a size distribution such that approximately 97% of particles have a size of greater than 4 μm; approximately 90% of particles have a size of greater than 10 μm; approximately 75% of particles have a size of greater than 20 μm; approximately 50% of particles have a size of greater than 25 μm; approximately 25% of particles have a size of greater than 30 μm; approximately 1% of particles have a size of greater than 70 μm.

7. The composition of claim 6 wherein the zirconium phosphate is obtained through a synthesis using a polyphosphate.

8. The composition of claim 7 wherein the synthesis includes zirconyl chloride.

9. The composition of claim 7 wherein the polyphosphate is selected from the group consisting of sodium hexametaphosphate, sodium trimetaphosphate, sodium tripolyphosphate, and sodium pyrophosphate.

10. The composition of claim 6 wherein the granules have a phosphate to a zirconium molar ratio of approximately 1.8/1 to about 2.2/1.

11. The composition of claim 6 wherein the granules have an ammonia sorption capacity of at least 0.8 mmol/g using an ammonia feed of approximately 7000 μmol/l.

12. A method of preparing zirconium phosphate particles comprising the steps of:
adding zirconyl chloride to a polyphosphate solution; and
heating a resultant solution or mixture above ambient temperature to obtain zirconium phosphate particles.

13. The method of claim 12 wherein the resultant solution or mixture was reduced by heating the solution or mixture under reflux to a temperature of at least 100° C.

14. The method of claim 12 wherein the zirconium phosphate particles are purified by a washing step.

15. The method of claim 12 wherein the pH of the polyphosphate solution is between approximately 3.8 to about 5.7 before addition of zirconyl chloride.

16. The method of claim 12 wherein the pH of the polyphosphate solution is between approximately 3.6 to about 5.5 before refluxing.

17. The method of claim 12 including a molar ratio of polyphosphate/zirconyl chloride of approximately 1/1 to about 10/1.

18. The method of claim 12 wherein the molar ratio of polyphosphate/zinconyl chloride is approximately 3/1 to about 5/1.

19. The method of claim 12 wherein the polyphosphate concentration is approximately 0.1 M to 1.5M.

20. The method of claim 12 wherein the polyphosphate is selected from the group consisting of sodium hexametaphosphate, sodium trimetaphosphate, sodium tripolyphosphate, and sodium pyrophosphate.

21. A composition including zirconium phosphate particles obtained from sodium pyropolyphosphate having a particle distribution such that approximately 97% of particles have a size of greater than 15 μm; approximately 90% of particles have a size of greater than 20 μm; approximately 75% of particles have a size of greater than 25 μm; approximately 50% of particles have a size of greater than 30 μm; approximately 25% of particles have a size of greater than 35 μm; approximately 1% of particles have a size of greater than 70 μm.

22. A composition including zirconium particles obtained from sodium triphosphate particles having a particle distribution such that at least 97% of the particles have a size of greater than 4 μm; at least 90% of the particles have a size of greater than 13 μm; at least 75% of the particles have a size of greater than 20 μm; at least 50% of the particles have a size of greater than 25 μm; at least 25% of the particles have a size of greater than 35 μm; and at least 1% of the particles have a size of greater than 84 μm

23. A composition for removing ammonia from a fluid stream the composition comprising particles of zirconium phosphate synthesized using polyphosphate and zirconium salt wherein the composition has an ammonia absorption capacity of at least 0.8 mmol/g using an ammonia feed of approximately 7000 μmol/l.

24. The composition of claim 23 wherein the polyphosphate is selected from the group consisting of sodium hexametaphosphate, sodium trimetaphosphate, sodium tripolyphosphate, and sodium pyrophosphate.

25. The composition of claim 23 wherein the granules have a phosphate to zirconium molar ratio of approximately 1.8/1 to about 2.2/1.

26. A particle bed for removing a component from a fluid stream comprising zirconium phosphate particles having a size distribution such that 97% of particles have a size of greater than 4 μm; approximately 90% of particles have a size of greater than 13 μm; approximately 75% of particles have a size of greater than 20 μm; approximately 50% of particles have a size of greater than 27 μm; approximately 25% of particles have a size of greater than 35 μm; approximately 1% of particles have a size of greater than 70 μm.

27. The particle bed of claim 26 wherein the zirconium phosphate is obtained through a synthesis using a polyphosphate.

28. The particle bed of claim 26 wherein the synthesis includes zirconyl chloride.

29. The particle bed of claim 26 wherein the polyphosphate is selected from the group consisting of sodium hexametaphosphate, sodium trimetaphosphate, sodium tripolyphosphate, and sodium pyrophosphate.

30. The particle bed of claim 26 wherein the granules have a phosphate to zirconium molar ratio of approximately 1.8/1 to about 2.2/1.

31. The particle bed of claim 26 wherein the granules include zirconium sodium pyrophosphate.

32. A method of providing dialysis comprising the step of passing a dialysate fluid through a particle bed including a composition comprising granules of zirconium phosphate synthesized using polyphosphate and zirconium salt that was prepared by mixing the polyphosphate and zirconium salt at a pH of at least 3 and heating to a temperature of greater than ambient conditions at a molar ration of 1/10 to 10/1.

33. The method of claim 32 wherein the polyphosphate is selected from the group consisting of sodium hexametaphosphate, sodium trimetaphosphate, sodium tripolyphosphate, and sodium pyrophosphate.

34. The method of claim 32 wherein the zirconium salt is selected from the group consisting of zirconyl chloride, zirconyl nitrate, and zirconium sulfate.

35. The method of claim 32 wherein the dialysis procedure is a continuous flow peritoneal dialysis procedure.
